(19) United States
(12) Patent Application Publication
(10) Pub. No.: US 2011/0287520 A1
(43) Pub. Date: Nov. 24, 2011

(54) SALMONELLA MARKER VACCINE

(76) Inventor: Heiko Apfel, Pulheim (DE)

(21) Appl. No.: 13/129,858

(22) PCT Filed: Nov. 20, 2009
(86) PCT No.: PCT/EP2009/065568

§ 371 Date: Aug. 3, 2011

(30) Foreign Application Priority Data
Nov. 20, 2008 (EP) 08020543.5

(51) Int. Cl. C12N 1/21; C12N 15/31; C07K 14/255

(52) U.S. Cl. 435/252.3; 530/324; 536/23.7

(57) ABSTRACT

The present invention relates to a Salmonella strain suitable as a marker vaccine having an inactivated gene.

The present invention relates to a Salmonella strain having an inactivated phoN gene. In particular, the invention relates to a method for the preparation of ΔphoN Salmonella enterica live-attenuated vaccine strains. Further, the invention relates to a serological test system which uses the recombinant Salmonella protein PhoN for discrimination of vaccinated animals from Salmonella infected ones. Furthermore a bacteriological test system is disclosed for DIVA.

Nontyphoidal Salmonellosis is a widespread disease in animals and humans. In men it is the most common food-borne disease which mainly originates from products of poultry and pigs. In the European Union a surveillance system will be established which should reduce the intake of pathogenic Salmonella ssp. into the food chain (regulation EC 2160/2003). Vaccination may reduce the prevalence of pathogenic Salmonella ssp. in livestock and thus minimize the transmission rate. Anti-Salmonella vaccines are commercially available e.g. for laying hens and pigs. The vaccines are either bacterins or live-attenuated Salmonella vaccines. In laying hens, these vaccines are used to reduce intra-ovarian transmission and fecal shedding of the pathogen.

The efficacy of live-attenuated Salmonella vaccines has been found superior to bacterins. The preferred use of live-attenuated Salmonella vaccines for livestock, demands a method for the Differentiation of infected from Vaccinated Animals (DIVA) which complies with the regulations of EC 2160/2003.

The majority of the authorized live-attenuated Salmonella vaccines are prepared from defined wildtype isolates of Salmonella enterica ssp. enterica serovars by chemical mutagenesis. Presently, live-attenuated Salmonella vaccines are distinguished from wildtype Salmonella ssp. by bacteriological means. These processes are long lasting and laborious. A serological discrimination of vaccinated and infected animals would be highly preferable. The presently available live-attenuated Salmonella vaccines do not enable a serological discrimination according to DIVA strategy.

DIVA live vaccines have been successfully used in livestock for the prevention of viral infections.

A marker vaccine can be prepared either as positive or negative marker. A positive marker vaccine contains an additional antigen which induces specific antibodies in vaccinated animals but not in infected ones. Positive marker vaccines are clearly disadvantageous because vaccinated animals with a subsequent infection cannot be discriminated from solely vaccinated ones.

A negative marker vaccine is prepared by removal of an antigen which provokes specific antibodies in infected animals. Thus, a negative marker vaccine provokes a humoral immune response in vaccinated animals that is different from that of infected animals. Ideally, this difference can be detected by serological means. In contrast to a positive marker vaccine, a negative marker vaccine enables the identification of vaccinated animals which become subsequently infected.

For the selection of a negative marker antigen several aspects must be considered: The antigen must be immunogenic but not be involved in the immune processes which protect animals against the pathogen. Thus the removal or inactivation of the gene which encodes the marker antigen must maintain immunogenicity of the vaccine strain. Finally, the marker antigen should be specific for the pathogen in order to avoid false-positive serological results which are induced by other organisms that may appear in livestock and provoke a humoral immune response.

Due to the high complexity of live-attenuated bacterial vaccines in contrast to viral vaccines much more effort is needed for the development of a live-attenuated bacterial marker vaccine. Live-attenuated bacterial marker vaccines are still at an experimental stage. The first candidates of a negative Salmonella marker vaccine were established from live-attenuated Salmonella vaccine strains by deleting the genes of highly immunogenic surface antigens. These live-attenuated Salmonella DIVA vaccines were able to differentiate infected from vaccinated animals by serological means. However, these prototypes were less protective than the parental vaccine strain.

The gene phoN encodes a non-specific acid phosphatase, which has been found widely preserved in all Salmonella ssp. The gene phoN is expressed under the control of the PhoPQ regulon, which is a general regulator for virulence genes in Salmonella. It is not known whether phoN is essential for the survival of Salmonella in animals.

The linkage of phoN to the PhoPQ regulon may indicate that phoN becomes activated whenever Salmonella occupies the host's tissue. Thus the question remains whether the elimination or inactivation of the gene phoN in a live-attenuated Salmonella vaccine strain may negatively affect its potential to induce protective immunity in vaccinated animals. It is known that the depletion of a single gene in a live-attenuated Salmonella vaccine strain may further reduce its residual virulence which may diminish the stimulation of the host's immune response and as a consequence may abrogate protective immunity.

It is the problem of the present invention to provide a Salmonella strain suitable as a negative marker vaccine which at least partially overcomes the above described disadvantages of state of the art vaccines.

In the present invention, the gene phoN has been selected as a target for the preparation of a Salmonella DIVA vaccine. Surprisingly, the phoN gene provides a combination of properties that make a phoN-deficient Salmonella strain especially suitable as a live vaccine.

The first surprising property is the different structure of the PhoN protein in all non-Salmonella bacteria compared with Salmonella. On the other hand, the PhoN amino acid sequence has a degree of at least 95% identity (calculated over the total length of the amino acid sequence) in different Salmonella species.

The second surprising property is the fact that PhoN is immunogenic. Analyses of sera from chicken and mice infected with Salmonella ssp. clearly revealed the appearance of antibodies which specifically bind to purified PhoN protein. This result provides the first evidence that PhoN is immunogenic and can be used as serological marker antigen for detecting Salmonella-infected birds and mammals. If the expression of the gene phoN is abolished in any Salmonella vaccine strain, this might allow the discrimination of infected animals from vaccinated ones by serological means. Thus, animals vaccinated by a Salmonella live vaccine strain with depleted PhoN antigen, will not produce PhoN-specific antibodies, whilst animals which become infected with wildtype Salmonella would do so.

The third surprising property is the fact that the removal of the gene phoN of a live-attenuated Salmonella vaccine strain may further reduce the virulence of the vaccine strain but does not affect its immunogenicity and its efficacy to protect vaccinated chicken from Salmonella infection of homologous serovar.

A first subject of the present invention is a Salmonella strain having an inactivated phoN gene. This in indicated herein by ΔphoN. A ΔphoN Salmonella strain has essentially no phoN activity. The person skilled in molecular biology knows methods of gene inactivation in bacteria such as Salmonella.

Inactivation of phoN includes deletion or/and modification of the phoN gene. In particular, the Salmonella strain of the present invention produces essentially no PhoN protein or a fragment thereof which is capable to induce an immune response against the PhoN protein or a fragment thereof, i.e. the Salmonella strain of the present invention is not immunogenic with respect to the PhoN protein or a fragment thereof. The phoN gene may be deleted completely or at least partially. Partial deletion of the phoN gene may be a deletion of at least 50%, at least 60%, at least 70%, at least 80%, at least 90%, or at least 95%, or at least 98% of the full-length sequence of the phoN gene. If the phoN gene sequence is deleted partially, the remaining sequences are preferably not be able to express a PhoN protein or a fragment thereof, in particular an immunogenic fragment as described herein.

Modification of the phoN gene sequence includes sequence replacement. The phoN gene may be completely replaced by another sequence. Deleted sequences of the phoN gene may be replaced completely or partially by other sequences.

The coding sequence (open reading frame) of the phoN gene may be deleted completely or at least partially. Partial deletion of the phoN coding sequence may be a deletion of at least 50%, at least 60%, at least 70%, at least 80%, at least 90%, or at least 95%, or at least 98% of the sequence of the full-length phoN coding sequence. If the phoN coding sequence is deleted partially, the remaining sequences are preferably not be able to express a PhoN protein or a fragment thereof, in particular an immunogenic fragment as described herein.

Modification of the phoN coding sequence includes sequence replacement. The phoN coding sequence may be completely replaced by another sequence. Deleted sequences of the phoN coding sequence may be replaced completely or partially by other sequences.

Typical examples of sequence of the phoN polypeptide in Salmonella are described in FIG. 1 (SEQ ID NO:1 to 20). Typical examples of the Salmonella phoN gene locus are described in FIG. 2 including the phoN open reading frames (ORF) and regulatory sequences upstream or/and downstream of the phoN ORF (SEQ ID NO:23 to 27).

Fragments of the phoN polypeptide of the present invention may have a length of a least 10, at least 20, or at least 30amino acid residues. They may have a length of at the maximum 200, at the maximum 150, or at the maximum 100 amino acid residues. Fragments include immunogenic fragments (also termed herein as immunogenic portions).

Inactivation of the phoN gene is preferably irreversible inactivation, such as by complete or at least partial deletion of the phoN coding region, or by replacement of the phoN coding region, as described herein. In particular, irreversible inactivation prevents the Salmonella strain of the present invention from reverting to the wildtype phoN genotype.

As indicated above, the Salmonella strain of the present invention produces essentially no PhoN protein which is capable to induce an immune response against the PhoN protein, i.e. the Salmonella strain of the present invention is not immunogenic with respect to the PhoN protein or a fragment thereof. Immunogenicity with respect to the PhoN protein or a fragment thereof may be an immunogenicity in any species to be treated with the Salmonella strain of the present invention. In particular, immunogenicity with respect to the PhoN protein or a fragment thereof is immunogenicity in mammals, e.g. pigs, or birds, e.g. poultry such as chickens.

The person skilled in the art is able to identify the PhoN polypeptide and the phoN gene in Salmonella or non-Salmonella strains. In the present invention, the Salmonella PhoN protein may be a protein comprising a sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO: 14, SEQ ID NO:15, SEQ ID NO:16, SEQ ID NO:17, SEQ ID NO:18, SEQ ID NO:19, and SEQ ID NO:20, or a polypeptide comprising a sequence which is at least 70%, at least 80%, at least 90%, at least 95%, at least 98%, or at least 99% identical to the selected sequence, wherein identity is calculated relative to the length of the selected sequence. A non-Salmonella PhoN protein may be a PhoN protein obtained from Escherichia or Shigella and may have a sequence selected from SEQ ID NO:21 and SEQ ID NO:22, or a polypeptide comprising a sequence which is at least 70%, at least 80%, at least 90%, at least 95%, at least 98%, or at least 99% identical to a sequence selected from SEQ ID NO:21 and SEQ ID NO:22, wherein identity is calculated relative to the length of the selected sequence.

The Salmonella strain which is subject to phoN inactivation as described herein in order to obtain a strain according to the present invention may be selected from Salmonella enterica ssp. It is preferred that the Salmonella strain is selected from serovars of Salmonella enterica ssp. enterica, for example from serovar Enteritidis, Dublin, Gallinarum, Typhimurium, Newport, Choleraesuis, Agona, Hadar, Heidelberg, Kentucky, Saintpaul, Virchow, Weltevreden, Javiana, Schwarzengrund, Paratyphi, and Typhi, or from the serovars listed in FIG. 1.

The Salmonella strain which is subject to phoN inactivation as described herein may be an attenuated Salmonella strain, for example a live-attenuated Salmonella strain, for example a live-attenuated Salmonella enterica ssp. enterica serovar Enteritidis His³¹ Ade⁻ strain such as the strain Salmovac SE (Springer et al., 2000, Berl. Münch. Tierärztl. Wschr. 113, 246-252) or a variant thereof.

Inactivation of the phoN gene as described herein may also be applied to authorized live-attenuated Salmonella vaccines. These strains may be transformed into live-attenuated Salmonella ΔphoN marker vaccines without affecting their immunogenicity in order to protect livestock e.g. from Salmonella infection and to simultaneously allow the discrimination of vaccinated from Salmonella infected livestock. This transformation method can be applied for live-attenuated Salmonella vaccine strains which have been prepared by chemical mutagenesis and/or genetic engineering, or for any recombinant Salmonella vaccine approach which uses the live-attenuated Salmonella vaccine strain for example as a carrier to express at least one additional autologous antigen and/or one additional antigen of a heterologous pathogen, or for live-attenuated Salmonella vaccine strain used as a carrier to deliver DNA for vaccination.

The Salmonella strain which is subject to phoN inactivation as described herein being a live-attenuated Salmonella vaccine strain is in particular
(i) a vaccine strain prepared by chemical mutagenesis and/or genetic engineering,
(ii) a recombinant vaccine strain which optionally recombinantly expresses at least one autologous Salmonella antigen and/or at least one heterologous antigen, e.g. an antigen of a heterologous pathogen, or
(iii) a recombinant carrier strain for the delivery of DNA.

The Salmonella strain of the present invention strain may be for use in medicine, e.g. in veterinary medicine. The Salmonella strain of the present invention may be for use as a vaccine. The Salmonella strain of the present invention may be for use as a live vaccine. The Salmonella strain of the present invention, in particular the vaccine as described herein may be for use as a vaccine to protect against a Salmonella infection, in particular against an infection with Salmonella enterica ssp. The Salmonella strain of the present invention may be for use in mammals, e.g. pigs, or birds, e.g. poultry such as chickens.

The Salmonella strain of the present invention strain may be used for the manufacture of a medicament, e.g. a medicament in veterinary medicine. The Salmonella strain of the present invention may be used for the manufacture of a vaccine. The Salmonella strain of the present invention may be used for the manufacture of a live vaccine. The Salmonella strain of the present invention, in particular the vaccine as described herein may be used for the manufacture of a vaccine to protect against a Salmonella infection, in particular against an infection with Salmonella enterica ssp. The Salmonella strain of the present invention may used for the manufacture of a medicament for use in mammals, e.g. pigs, or birds, e.g. poultry such as chickens.

The vaccine of the present invention may be a vaccine to protect against subsequent Salmonella infection, in particular against a subsequent infection with Salmonella enterica ssp., and to prepare polyvalent recombinant Salmonella carrier vaccines which protect against additional pathogens. The vaccine optionally recombinantly expresses at least one autologous Salmonella antigen and/or at least one heterologous antigen, e.g. an antigen of a heterologous pathogen. The vaccine may be suitable for administration to a mammal, e.g. a pig, or birds, e.g. poultry such as chickens.

Subject of the present invention is a method for prevention or/and treatment of a Salmonella infection comprising administration of a Salmonella strain of the present invention to a subject in need thereof. The subject may be a mammal, e.g. a pig, or birds, e.g. poultry such as chickens.

A further subject of the present invention is a method of prevention or/and treatment of an infection with a pathogen comprising administration of a Salmonella strain of the present invention to a subject in need thereof, wherein the Salmonella strain of the present invention is prepared to confer protection against this pathogen, for instance a polyvalent Salmonella live vaccine. The subject may be a mammal, e.g. a pig, or birds, e.g. poultry such as chickens. In particular, the pathogen is different from Salmonella.

Yet another subject of the present invention is a method for production of Salmonella strain as described herein, comprising inactivating the phoN gene of a Salmonella strain.

As indicated above, inactivation of phoN may further reduce the virulence of a live-attenuated Salmonella vaccine strain, but it does not affect the immunogenicity of that transformed Salmonella vaccine strain. Therefore phoN inactivation is a suitable method to produce live-attenuated Salmonella DIVA vaccine strains.

The preparation of a live-attenuated ΔphoN Salmonella DIVA vaccine may start from any live-attenuated Salmonella vaccine strain which is able to protect for instance avian and/or mammal livestock against a Salmonella infection, in particular against an infection with Salmonella enterica ssp. From such a Salmonella vaccine strain the gene phoN is inactivated as described herein by applying genetic engineering. After completion of the inactivation of phoN, the individual Salmonella variant strains have to be analyzed in order to evaluate differences which appear in relation to the parental Salmonella vaccine strain. The ΔphoN Salmonella variant strain are preferably essentially identical in the biological characteristics except phoN from the parental Salmonella vaccine strain. If the ΔphoN Salmonella variant strain differs from the parental Salmonella vaccine strain in one or more characteristics not related to phoN, these alterations may be permanent and, most importantly, may essentially maintain the original immunogenic character of the parental strain which provokes in appropriately vaccinated animals a protective immune response against subsequent Salmonella infection.

Preferentially, phoN is inactivated irreversibly by deletion of the nucleic acid fragment encoding the gene phoN by means of genetic engineering. The inactivation process may also include the deletion of accessory nucleic acids from which the transcription of phoN is initiated. The deletion of phoN may be performed in such a manner which suppresses the appearance of novel transcripts. Genetic engineering encompasses any processes of homologous recombination which enable the deletion of the gene phoN in the genome of Salmonellae as indicated herein. Preferably, the process of homologous recombination should enable the precise deletion of the gene phoN whilst the rest of the genome remained unchanged, which is designated herein as “clean” deletion process. Furthermore a process of homologous recombination is preferred which is driven by a temporarily co-expressed recombinase, such as the phage λ Red recombinase system as described by Datsenko and Wanner.

The Salmonella strain of the present invention may have a reduced motility, e.g. by at least 30%, at least 50% or at least 90%, or/and a reduced biofilm forming capability, e.g. by at least 30%, at least 50% or at least 90%.

As indicated above, the ΔphoN Salmonella strain, in particular the live-attenuated ΔphoN Salmonella strain may be a marker vaccine. Live-attenuated ΔphoN Salmonella marker vaccine strains enable the Differentiation of infected from Vaccinated Animals (DIVA) by serological and/or supplementary bacteriological means.

Yet another subject of the present invention is the use of a polypeptide comprising a Salmonella PhoN polypeptide or/and an immunogenic fragment thereof as a serological marker antigen. The serological marker antigen may be any antigen derived from the PhoN polypeptide. The phoN antigen may be a polypeptide comprising the full-length PhoN polypeptide, or comprising any immunogenic portion thereof which has a length of preferably at least 10, more preferably at least 20 and even more preferably at least 30 amino acids. The PhoN polypeptide may be a polypeptide comprising a sequence selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO: 14, SEQ ID NO:15, SEQ ID NO:16, SEQ ID NO:17, SEQ ID NO:18, SEQ ID NO:19, and SEQ ID NO:20, or a polypeptide comprising a sequence which is at least 70%, at least 80%, at least 90%, at least 95%, at least 98%, or at least 99% identical to a sequence selected from SEQ ID NO:1, SEQ ID NO: 2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO: 17, SEQ ID NO:18, SEQ ID NO:19, and SEQ ID NO:20, wherein % identity is calculated relative to the full length of the selected sequence. The immunogenic portion may have a sequence having at least 70%, at least 80%, at least 90%, at least 95%, at least 98%, or at least 99% identical amino acid positions to the sequence of a PhoN polypeptide described herein, wherein % identity is calculated relative to the length of the immunogenic portion. Identity as described herein may be calculated by common algorithms such as BLAST or FASTA. A preferred sequence is SEQ ID NO: 1 derived from strain CLAB_SE 360 (see FIG. 1).

The serological marker antigen as described herein may be used for the differentiation of infected from vaccinated animal (DIVA) after vaccination with the Salmonella strain of the present invention. A serological marker antigen derived from SEQ ID NO:1 may in particular be used for differentiation of an infected animal from a vaccinated animal.

The phoN antigen of the present invention may be prepared by recombinant expression of a phoN nucleic acid, particularly in a recombinant host cell such as E.coli. The phoN antigen may be provided as a frozen stock or as a lyophilisate, optionally together with appropriate preservatives.

Yet another subject of the present invention is a serological test system for detecting antibodies directed against a Salmonella phoN antigen, comprising at least one recombinant Salmonella phoN antigen or any immunogenic portion thereof, and optionally further test components. The serological test system may be capable of discrimination of Salmonella infected animals from animals vaccinated by a Salmonella strain of the present invention, in particular a live-attenuated ΔphoN Salmonella DIVA vaccine. The at least one phoN antigen is an antigen as described herein. The Salmonella phoN antigen preferably comprises the sequence SEQ ID NO:1 or a sequence which is at least 70%, at least 80%, at least 90%, at least 95%, at least 98%, or at least 99% identical to SEQ ID NO:1, wherein identity is calculated relative to the length of SEQ ID NO:1.

In the serological test system, the antigen serves as a target antigen which specifically detects antibodies in the serum of animals suspected of being infected with Salmonella or may become infected with Salmonella after vaccination. No antibodies are detected by the PhoN target antigen if the serum of animals is used which have been successfully immunized by a Salmonella strain of the present invention, in particular a live-attenuated ΔphoN Salmonella DIVA vaccine. Detection may be performed in the ELISA format.

Optionally an additional Salmonella specific antigen is used in the serological test system, such as Salmonella LPS, which detects antibodies induced by both wildtype Salmonella and the Salmonella vaccine strain of the present invention, in particular the live-attenuated ΔphoN Salmonella DIVA vaccine.

Yet another subject of the present invention is a phoN nucleic acid encoding a Salmonella antigen as described herein. The phoN nucleic acid comprises a sequence encoding a PhoN polypeptide or a fragment thereof as described herein, for instance a polypeptide comprising a sequence selected from SEQ ID NO:1, SEQ ID NO:2, SEQ ID NO:3, SEQ ID NO:4, SEQ ID NO: 5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO: 15, SEQ ID NO:16, SEQ ID NO:17, SEQ ID NO:18, SEQ ID NO:19, and SEQ ID NO:20, or a polypeptide comprising a sequence which is at least 70%, at least 80%, at least 90%, at least 95%, at least 98%, or at least 99% identical to said sequence, wherein identity is calculated relative to the length of said sequence. The nucleic acid may code for any immunogenic fragment of a PhoN polypeptide as described herein.

The phoN nucleic acid may be used for the manufacture of a recombinant Salmonella phoN antigen or any immunogenic portion thereof, e.g. in a host such as E. coli by using an appropriate recombinant DNA vector. The phoN antigen may be an antigen as described herein. The phoN antigen may comprise the sequence SEQ ID NO:1, or a sequence which is at least 70%, at least 80%, at least 90%, at least 95%, at least 98%, or at least 99% identical to SEQ ID NO:1, or any immunogenic portion thereof, as described herein. Identity is calculated relative to the length of SEQ ID NO:1.

A further subject of the present invention concerns a bacteriological test system for the differentiation of wildtype Salmonella ssp. and a Salmonella strain of the present invention, such as a live-attenuated ΔphoN Salmonella DIVA vaccines. This bacteriological test system is completing the official standard methods which regulate the detection of Salmonella in biological samples.

The inventive bacteriological test system clarifies whenever Salmonella colonies appear in the standard detection methods whether these are wild-type Salmonella or Salmonella strain of the present invention, such as a live-attenuated ΔphoN Salmonella. At least one suspicious Salmonella colony is analyzed in order to evaluate whether the genome of the individual Salmonella colonies contains the gene phoN or a fragment thereof. Analysis may be performed by a PCR method, or/and by detection of PhoN activity or/and a PhoN gene product. A representative number of suspicious Salmonella colonies may be analyzed, for instance at least 5, at least 10, at least 15, or at least 20 colonies.

PhoN activity can be measured by transferring aliquots of single colonies into separate vials, such as a well of microtiter plate, which contain a PhoN specific substrate mix where PhoN generates a chromogenic product that can be detected by visual control or by an appropriate device. PhoN is localized in the periplasmic space of Salmonella ssp which allows the use of intact cells for determination of PhoN activity. According to the test system wildtype Salmonella show PhoN activity by generating a chromogenic product whereas live-attenuated ΔphoN Salmonella do not. A suitable substrate may be BCIP.

The Salmonella strain of the present invention may be detected by a test according to ISO 6579. ISO 6579 is included herein by reference. By a test according to ISO 6579, PhoN activity may be detected by the chromogenic substrate BCIP.

The invention is further illustrated by the following Figures, Examples and the sequence listing.

### FIGURE LEGENDS

FIG. 1: Similarities of PhoN proteins between various Salmonella enterica enterica serovars.

FIG. 2: The genomic regions of phoN of Salmonella enterica enterica serovars.

FIG. 3

FIG. 3A schematically shows the genomic region of S. Enteritidis P125109 where the gene phoN is localized. The illustrated genomic region starts at nucleotide (nt) 4.419.410 of the sequence NC_{—}011294.1 disposed at the NCBI data base. The orientation and length of any open-reading-frame (ORF) is indicated by arrows. Annotated ORFs are illustrated by black arrows and the others by striped arrows. For phoN, two promoters (P1, P2) have been predicted which are illustrated by open arrows. Reverse transcriptase-PCR analyses revealed that transcription of phoN is most probably initiated at P1. The preferred phoN deletion region is indicated by solid vertical bars and the flanking regions, which are used for the genetic engineering processes, by dashed bars.

FIG. 3B schematically illustrates clean phoN deletion within the genome of S. Enteritidis P125109 after applying genetic engineering processes as indicated.

FIG. 4

FIG. 4 shows the primary structure of a DNA-fragment which may be used for the genetic engineering process as outlined in FIG. 1, leading to clean deletion of phoN in a given Salmonella Enteritidis vaccine strain having identical primary structure. The flanking regions are indicated.

FIG. 5

FIG. 5 exemplarily shows the strategy for the preparation of phoN deletion mutant of Salmonella enterica enterica Enteritidis strain P125109 by replacement recombination. FIG. 5A schematically shows the genomic region of S. Enteritidis strain P125109 as indicated in the legend to FIG. 3.

FIG. 5A exemplarily illustrates the boundary (solid vertical lines) of a deletion region which comprises the gene phoN. FIG. 5B illustrates the ORFs within the original genomic region after replacement of phoN. Three of the original ORFs (ORF1-3) become extended by the recombination process (open arrow).

FIG. 6

FIG. 6 illustrates RT-PCR analyses within the genomic region of a phoN mutant strain prepared from S. Enteritidis vaccine strain Salmovac SE by replacement recombination as outlined in FIG. 5.

In section A, data of RT-PCR analyses of the ORF designated as putative acetyltransferase (black arrow) are illustrated. The analyses revealed a transcript (mRNA) that encompasses the putative acetyltransferase and the following ORFs 1-3. The RT-PCR analyses further revealed that the transcript extends into the replacement fragment but, surprisingly, does not pass that fragment.

In the upper right frame, the primary structure of the replacement fragment and the included FRT-site is shown. The FRT-site forms a dyad structure of high stability (—17 kcal/mol). It is suggested that the FRT-site actually acts as a transcriptional terminator which supresses the expression of the ORFs 1-3 beyond the replacement fragment.

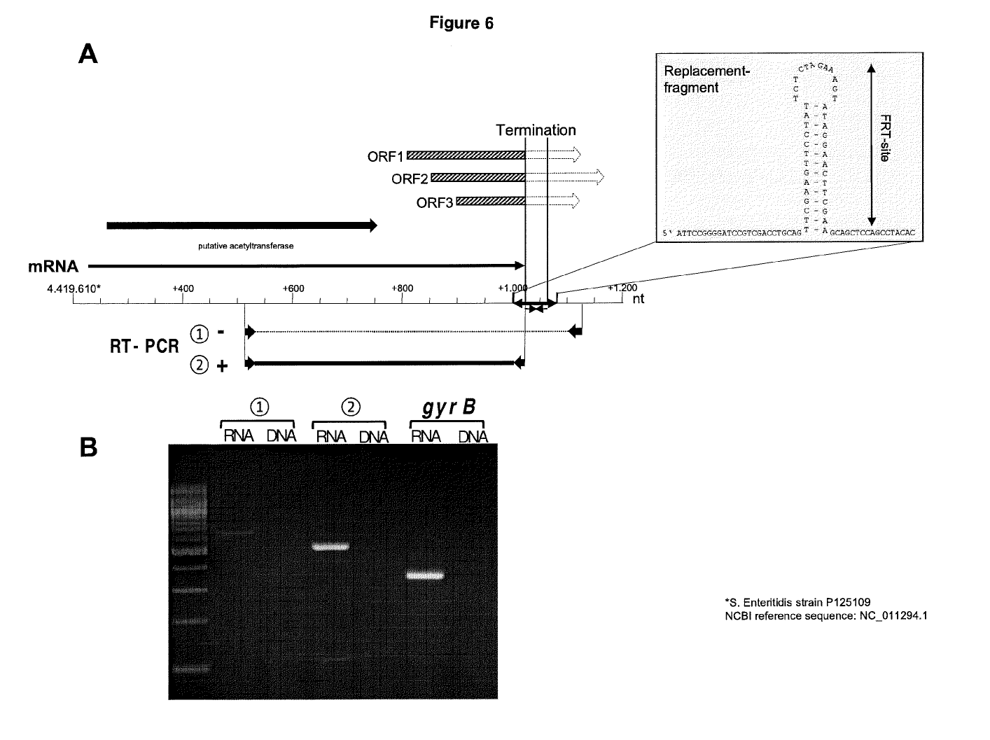

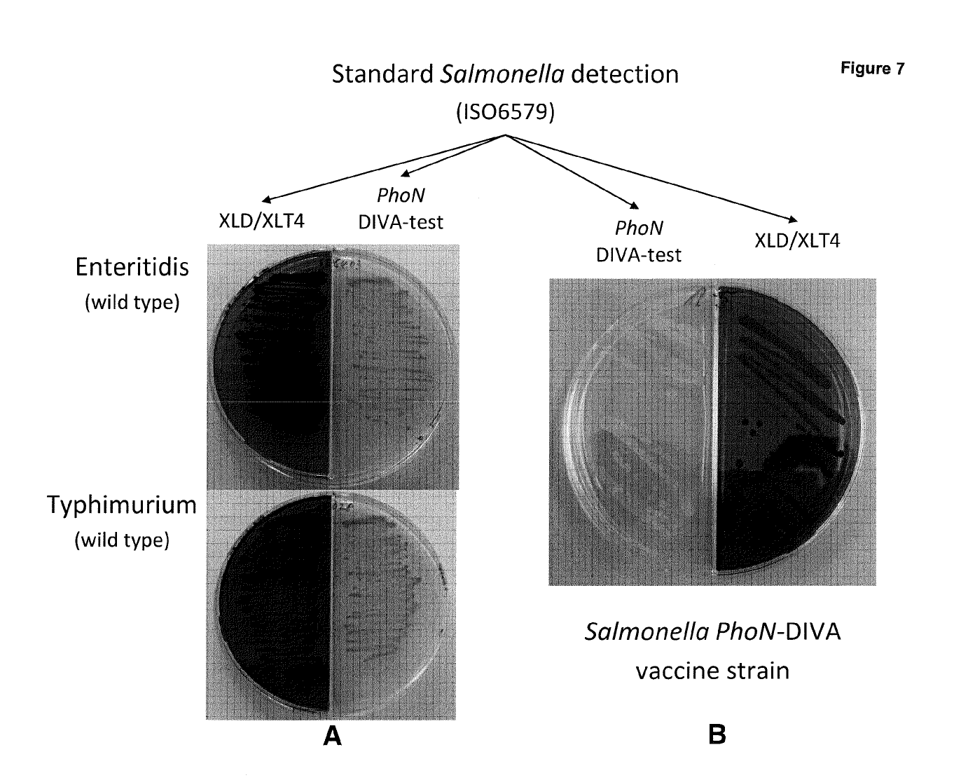

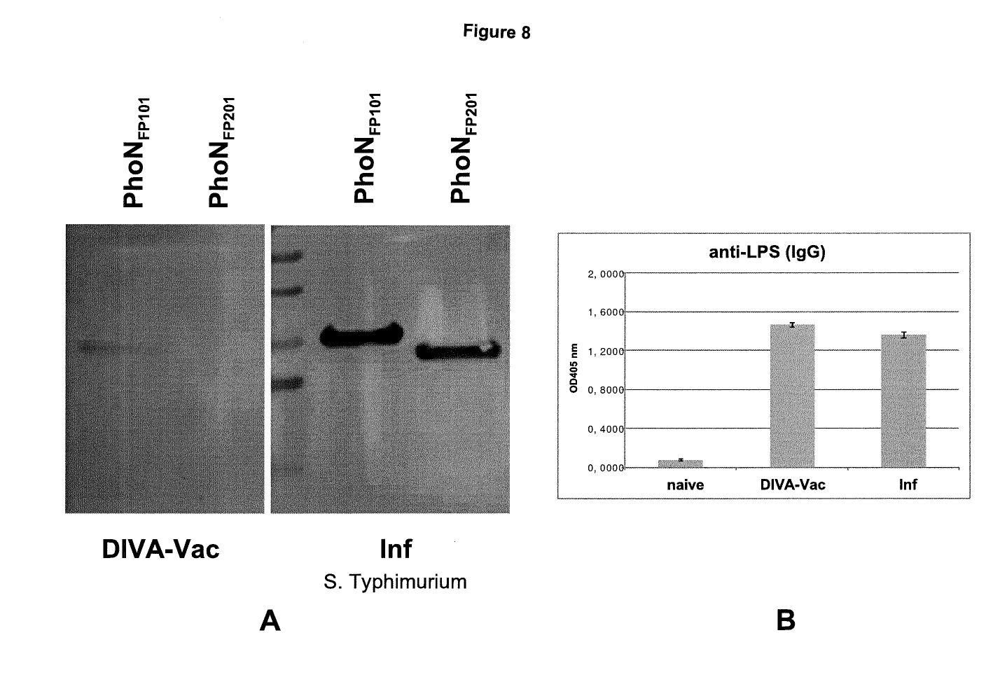

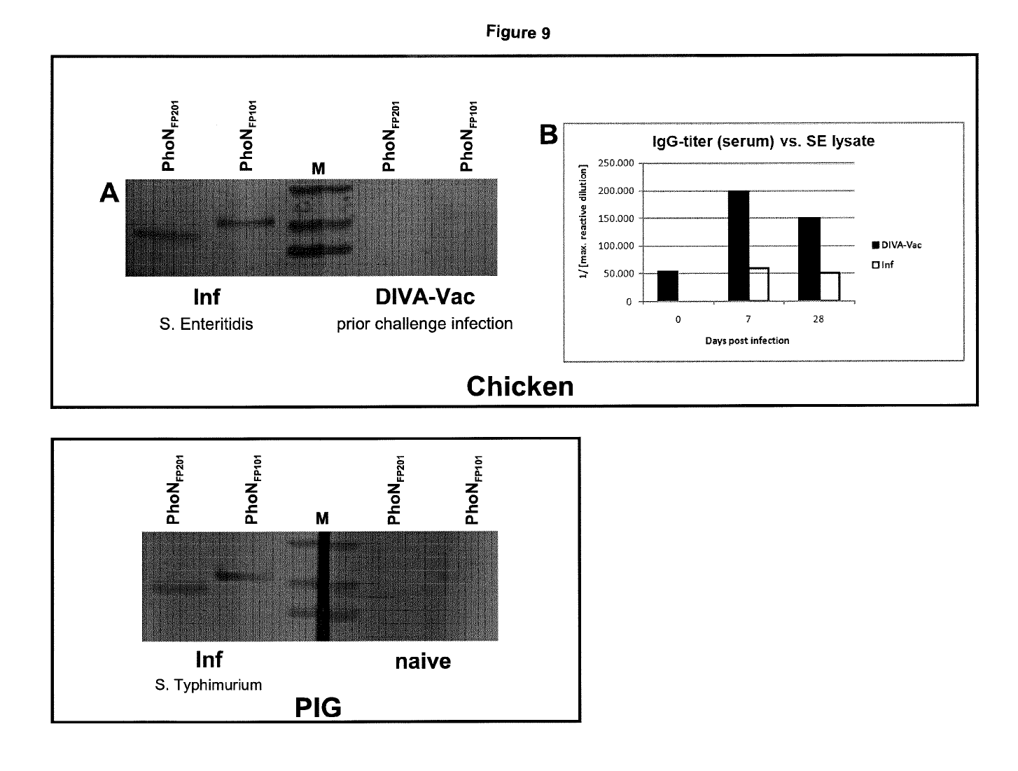

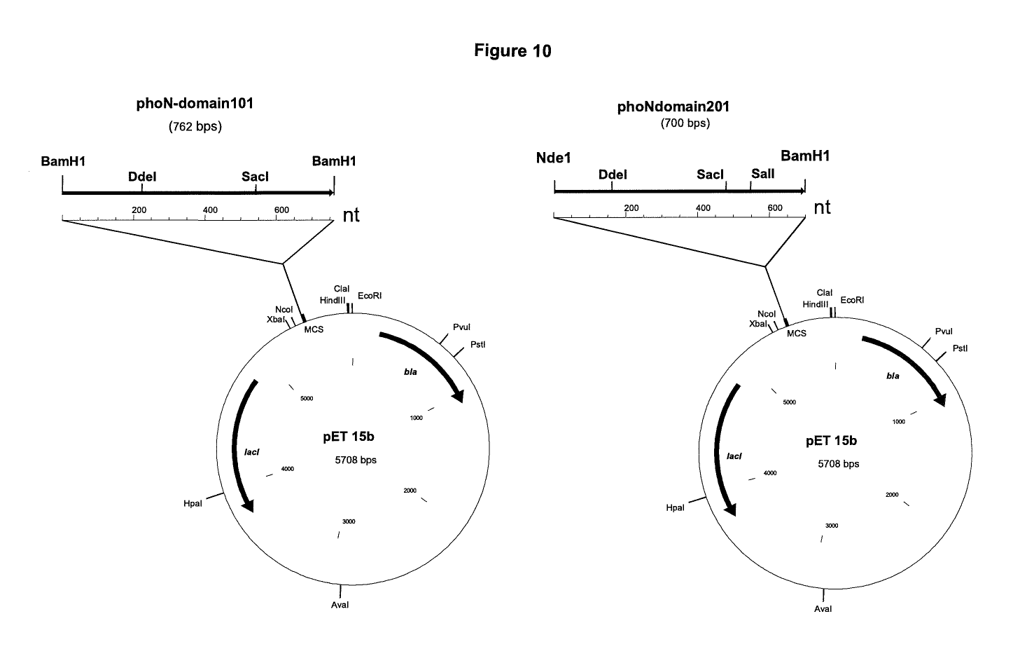

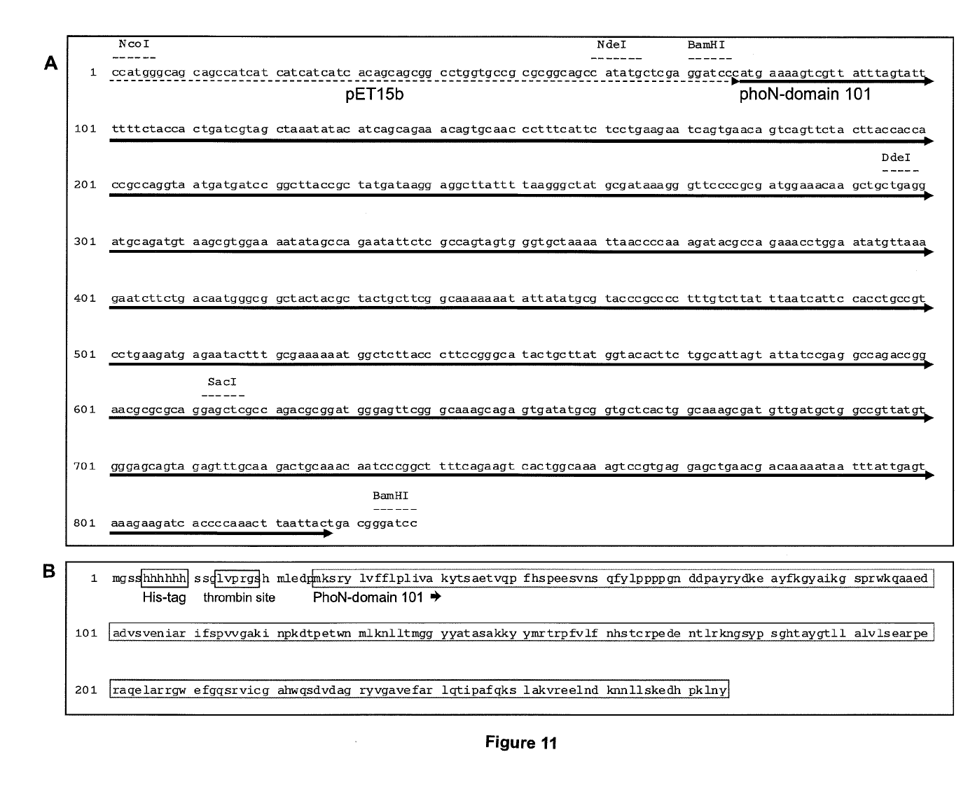

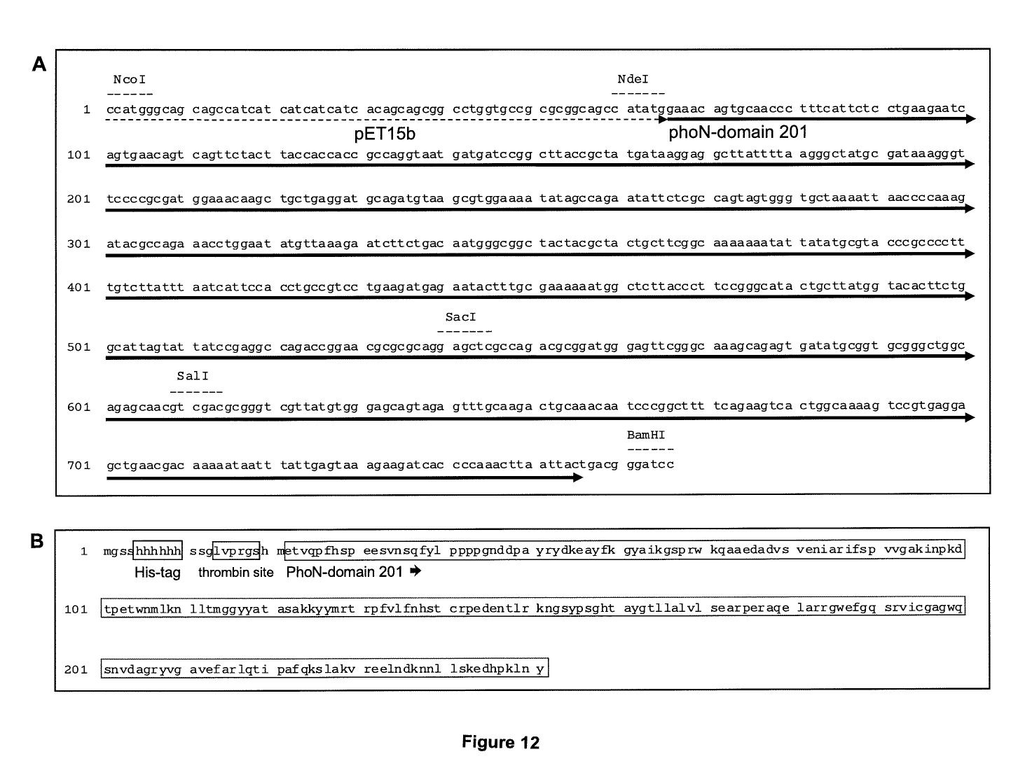

The primers for the RT-PCR are conceived to detect transcripts which either pass through the replacement fragment (1) or which cover the region in front of the FRT-site (2). FIG. 6B shows agarose gel electrophoresis of the RT-PCR products obtained from RNA preparations of S. Enteritidis vaccine strain which have been treated with reverse transcriptase (RNA) or remained untreated (DNA) before PCR. The data reveal pure RNA probes. As a control, the RT-PCR of the gene gyrB is presented. As already indicated, the transcript beyond the replacement fragment is almost abolished (lane 1) which is in contrast to the transcript that ends in front of the FRT-site (lane 2).

FIG. 7

Approval of the Bacteriological phoN-DIVA Test which is Used in Parallel to Standard Salmonella Detection Test According to ISO6579.

FIG. 7A exemplarily illustrates parallel plating of wild-type Salmonella Typhimurium and Enteritidis, respectively, on XLT4 agar plates and on LB-agar plates supplemented with the chromogenic phosphatase substrate BCIP [80 μg/mL]. Wild-type Salmonellae Typhimurium and Enteritidis generally appear on XLD/XLT4 agar plates as black colored colonies and on BCIP LB-agar plates (PhoN-DIVA test) as blue-green colored colonies.

In FIG. 7B the parallel plating of a live attenuated Salmonella Enteritidis phoN-DIVA vaccine strain is shown. PhoN-DIVA Salmonella vaccine strain appears as black-colored colonies on Salmonella specific XLD/XLT4 agar plates but remained uncolored on BCIP LB-agar plates.

The outlined testing allows clear differentiation of infected from vaccinated animals within the standard Salmonella test set-up.

FIG. 8

Specificity of PhoN-DIVA antibody test as approved by immunoblot analysis using sera from Salmonella-infected mice (Inf) and sera from mice immunized with live-attenuated Salmonella phoN-DIVA vaccine strain (Vac). The blots of graph A were prepared with two recombinant variants of Salmonella PhoN, PhoN_{FP101} and PhoN_{FP201}. The purified samples have been separated on 10% Schägger-Jagow PAGE prior blotting. PhoN_{FP101} covers the primary structure of the original PhoN. PhoN_{FP201} has truncated NH2-terminus and amino acids substitutions within the active center of PhoN yielding an inactive PhoN. The immunoblot analyses show IgG-specific immune response using appropriate alkaline phosphatase-conjugated antisera. Molecular weight markers are shown in lane M, corresponding to 55, 43, 34 and 26 kDa.

Graph A presents a PhoN-DIVA antibody test with sera from mice immunized with live attenuated S. Typhimurium SL1344 ΔaroA phoN-DIVA vaccine strain (Vac) and sera from mice (Nramp⁺) infected with virulent progenitor strain S. Typhimurium SL1344 (Inf). The antibodies of the Inf-group bind to both variant PhoN antigens. The Vac-group does not produce PhoN-specific antibodies. As shown by LPS-ELISA (B), both, the Vac- and Inf-group produce almost identical quantities of S. Typhimurium LPS-specific antibodies.

FIG. 9

PhoN-DIVA Antibody Test in Chickens and Pigs.

In the upper graph, the serological data of chickens are presented which either became orally infected with wild type S. Enteritidis strain (Inf) or were orally immunized with live attenuated S. Enteritidis phoN-DIVA vaccine strain (DIVA-Vac). In A, the immunoblot data of both animal groups is presented. In B, the IgG-titer against S. Enteritidis lysate is illustrated as determined in an ELISA using sera of both animal groups at different times of infection. In the DIVA-Vac group, IgG-titer was determined before (0) and after challenge infection, at days 7 and 28 post infection. In the Inf group the IgG-titer was determined in the same manner.

In the lower graph, the immunoblot analysis of S. Typhimurium infected pigs and their naïve counterparts is presented.

The immunoblots were prepared as outlined in the legend to FIG. 8.

FIG. 10

Maps of the expression plasmids used for the preparation of the fusion proteins PhoN_{FP101} (left-hand map) and PhoN_{FP201} (right-hand map).

FIG. 11

Sequence of the gene (A) and the encoded fusion protein (B) of PhoN_{FP101}.

FIG. 12

Sequence of the gene (A) and the encoded fusion protein (B) of PhoN_{FP201}.

### EXAMPLE 1

The similarities of the protein PhoN has been assessed within the various Salmonella enterica enterica serovars by BLAST 2.2.18 using the database “nr” via the Internet portal of the National Center for Biotechnology Information (NCBI). The amino acid sequence of the protein PhoN of the SalmonellaEnteritidis strain CLAB_SE360 has been used as query sequence (SEQ ID NO:1). CLAB_SE360 is a variant of the vaccine strain Salmovac SE (Springer et al., 2000, Berl. Münch. Tierärztl. Wschr. 113, 246-252). Non-identical amino acids within the primary structure of the various PhoN proteins are highlighted by color. The data reveal that the primary structures of the PhoN protein within the various Salmonella serovars have an identity which is >96%.

In contrast, the primary structures of the PhoN protein within the non-Salmonella bacteria have low relationship (identity<40%) with the PhoN protein of Salmonella enterica enterica serovars. The most related PhoN amino acid sequences of non-Salmonella bacteria are shown. Only identical amino acids are displayed. Variant (−), additional (+) or missing (Δ) amino acids are indicated. The full sequences are given in the sequence listing (SEQ ID NO:21 and 22).

The results are summarized in FIG. 1.

### EXAMPLE 2

Description of the phoN genomic regions of various Salmonella enterica enterica serovars comprising the gene phoN and the flanking regions which are of relevance for the preparation of ΔphoN Salmonella mutants by genetic engineering, such as described by T. Shigaki and K. D. Hirschi (Anal. Biochem. 2001, 298:118-120) or by K. A. Datsenko and B. L. Wanner (PNAS 2000, 97:6640-6645).

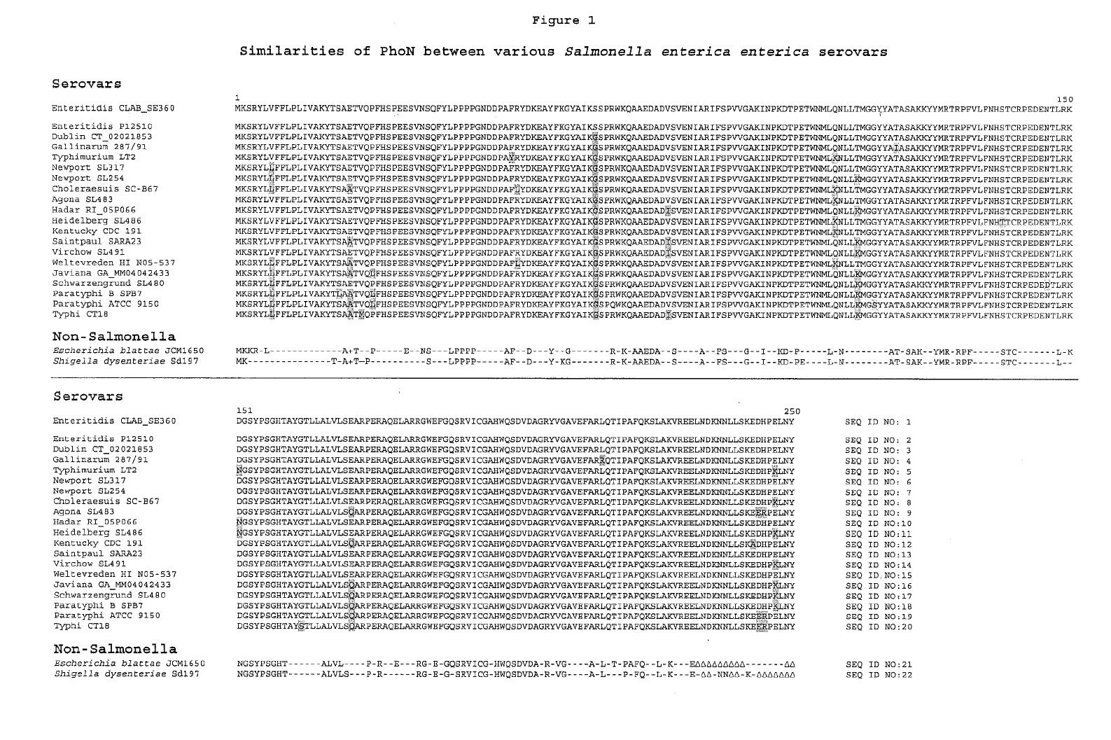

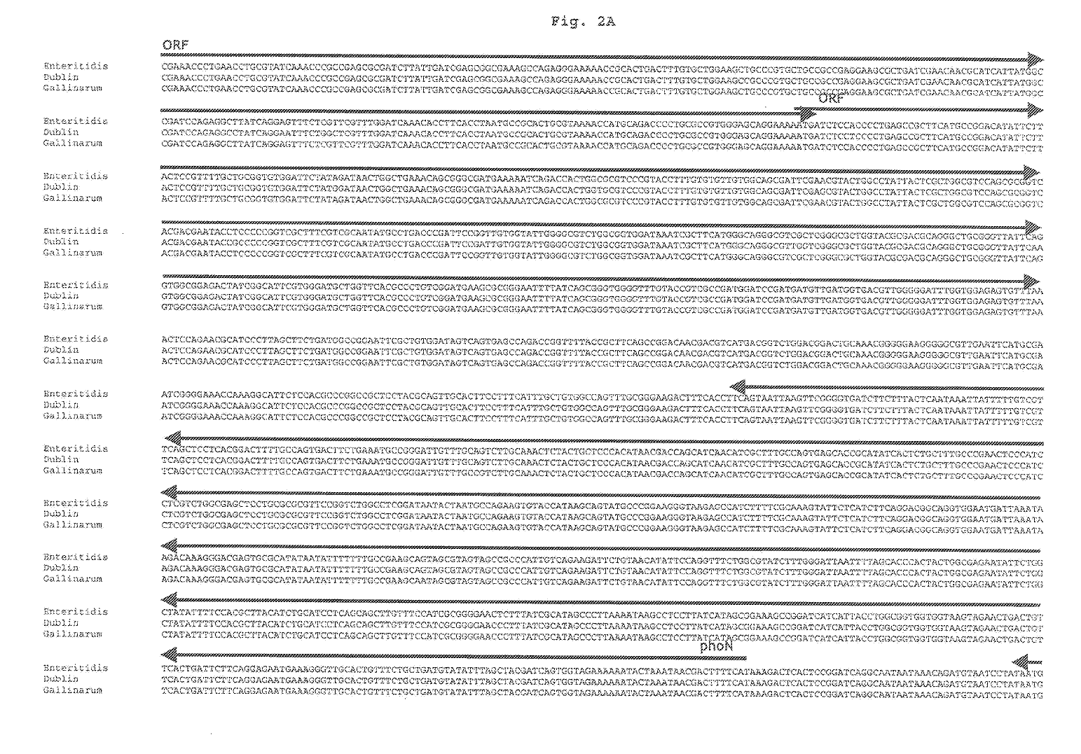

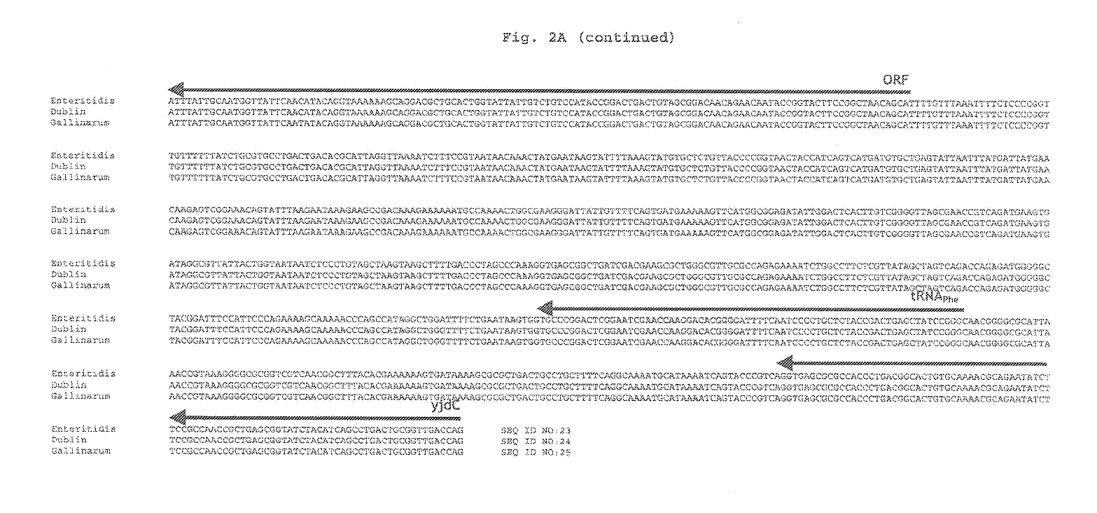

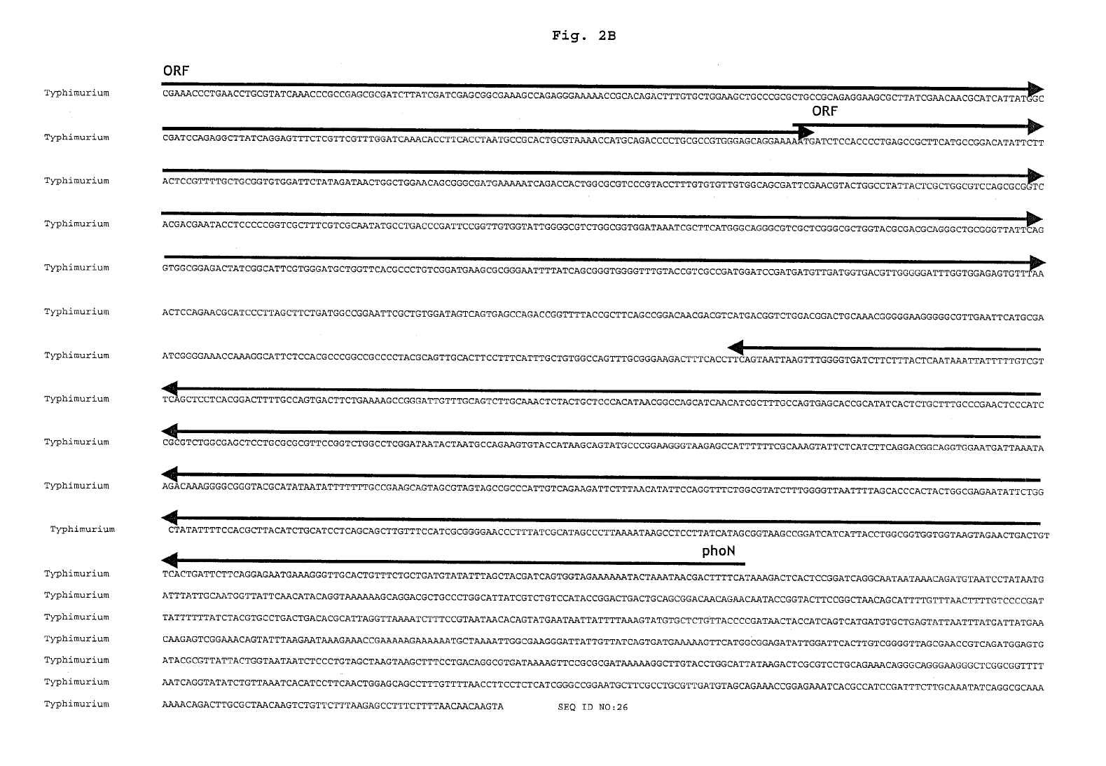

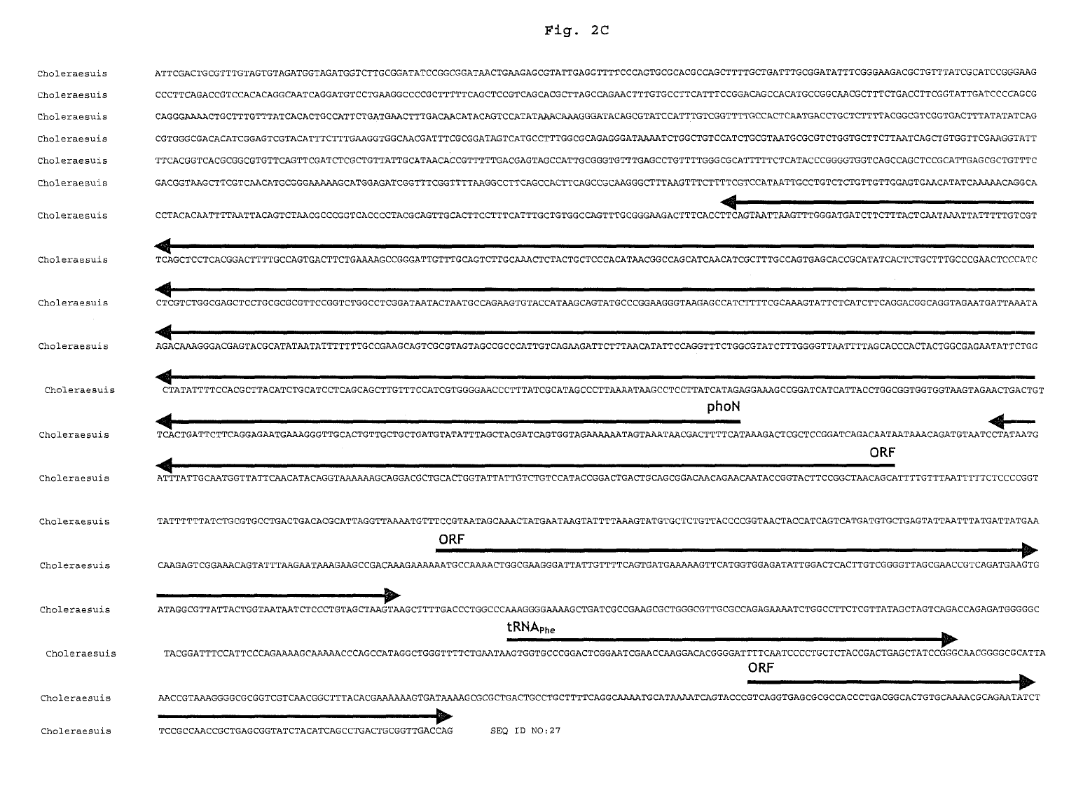

The phoN genomic regions are shown in FIG. 2 as follows:
FIG. 2A S. Enteritidis str. P125109 (excerpt from RefSeq NC011294.1) S. Dublin str. CT_{—}02021853 (excerpt from RefSeq NC011205.1) S. Gallinarum str. 287/91 (excerpt from RefSeq NC011274.1)
FIG. 2B S. Typhimurium str. LT2 (excerpt from RefSeq NC003197.1)
FIG. 2C S. Choleraesuis str. SC-B67 (excerpt from RefSeq NC006905.1)

### EXAMPLE 3

Preparation of Live-Attenuated Salmonella enterica enterica phoN-DIVA Vaccine Strain.

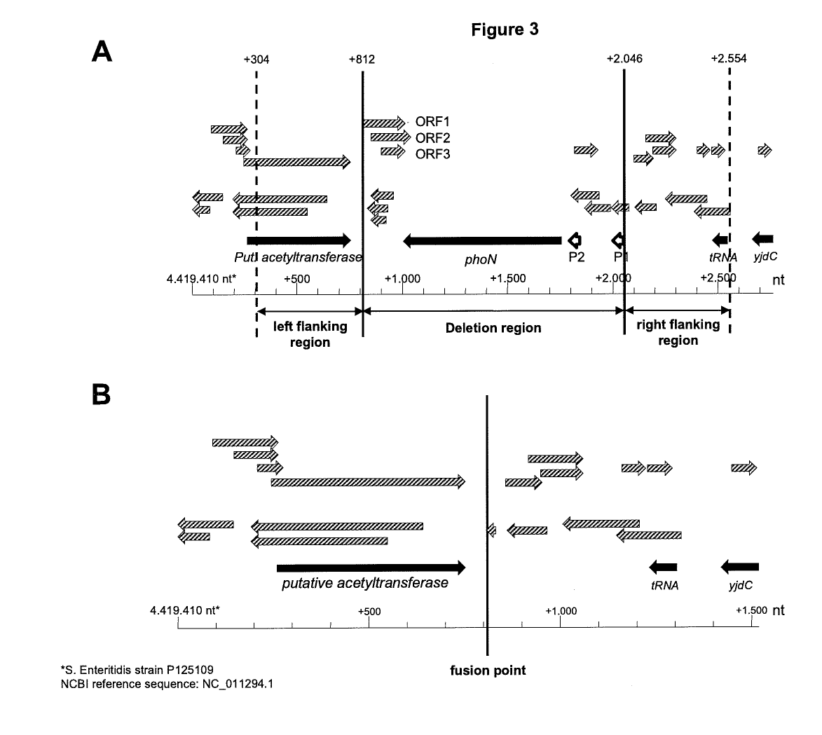

FIG. 3 exemplarily shows the strategy for the preparation of a clean phoN deletion mutant Salmonella vaccine strain using the genomic sequence data of Salmonella enterica enterica Enteritidis strain P125109 which are accessible at the NCBI data base (http://www.ncbi.nlm.nih.gov/genomes). The genomic region adjacent to phoN contains several potential ORFs. Reverse transcriptase-PCR (RT-PCR) analysis of isolated mRNAs revealed that the putative acetyltransferase gene is transcribed in S. Enteritidis. Moreover, this transcript also includes ORFs 1-3 which finally end within phoN. Accordingly, these ORFs are included in the deletion process. In addition, the deletion process includes the transcriptional promoter of phoN. The deletion of the promoter avoids the appearance of novel transcripts and unexpected gene product after removal of the gene phoN. Two potential promoters have been predicted for phoN. RT-PCR analysis indicated a phoN transcript that starts from the most distant promoter P1. This promoter is localized in front of an ORF which is expected to be non-functional and disposable.

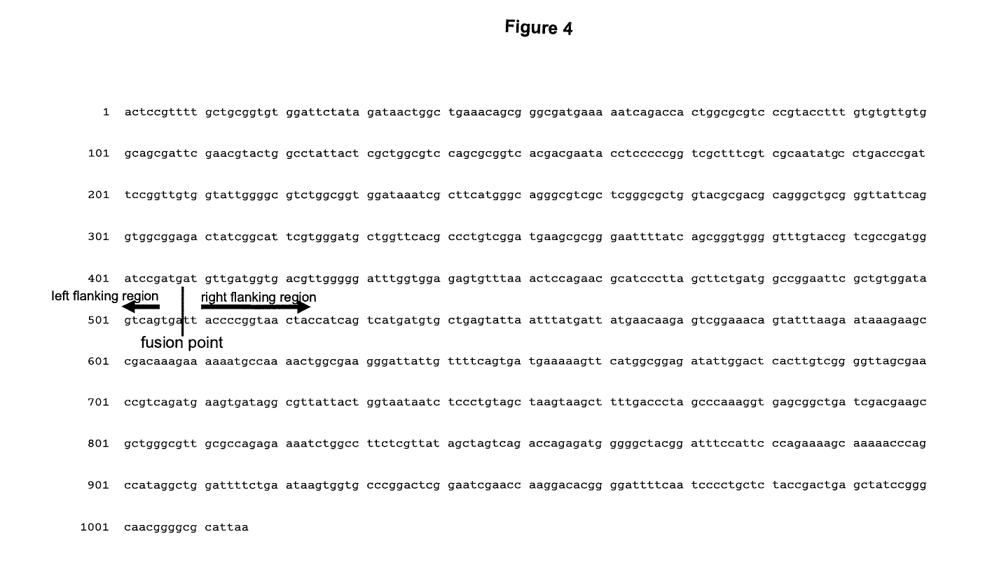

FIG. 3A exemplarily shows the boundary (solid vertical bars) of a deletion region for the clean removal of phoN as indicated in the previous paragraph. The flanking genomic regions on the left (nt 304-812) and right (nt 2.046-2.554) side of the deletion region are conceived for the genetic engineering process which ends up in the fusion of both flanking regions as illustrated in FIG. 3B, generating a clean deletion of phoN. The primary structure of a DNA-fragment which may be used for the genetic engineering process leading to clean deletion of phoN in S. Enteritidis strain P125109 is disclosed in FIG. 4. Such a DNA fragment may be prepared by PCR using genomic DNA of the appropriate Salmonella vaccine strain as a template. Afterwards, the Salmonella vaccine strain may be transformed with the purified DNA fragment or with a plasmid which contains the DNA fragment. Preferably a plasmid is used which does not replicate after transformation of Salmonella. Most preferably, the plasmid conditionally replicates in Salmonella. The introduced DNA fragment replaces the original genomic fragment by the cellular processes. However, the cellular processes of homologous recombination may be further improved by a temporarily co-expressed recombinase, such as the phage λ Red recombinase system as described by Datsenko and Wanner.

Alternatively, phoN may be replaced by DNA-fragment which contains accessory structures that suppress expression of adjacent ORFs. Replacement recombination is preferentially performed by the Red Phage system as described by Datsenko and Wanner. This method strongly facilitates the recombination process by extra recombinases which are introduced by temporarily replicating plasmids, e.g. pKD46. According to the method of Datsenko and Wanner, the replacement-fragment contains at least a structure (FRT site) which is used by the site-specific recombinase FLP. Ideally, the replacement-fragment further contains stop codons which abrogate translation in all 3 reading frames of both, the (+) and the (−) strand. More ideally, the replacement-fragment contains a transcriptional terminator which abrogates expression within the replacement fragment.

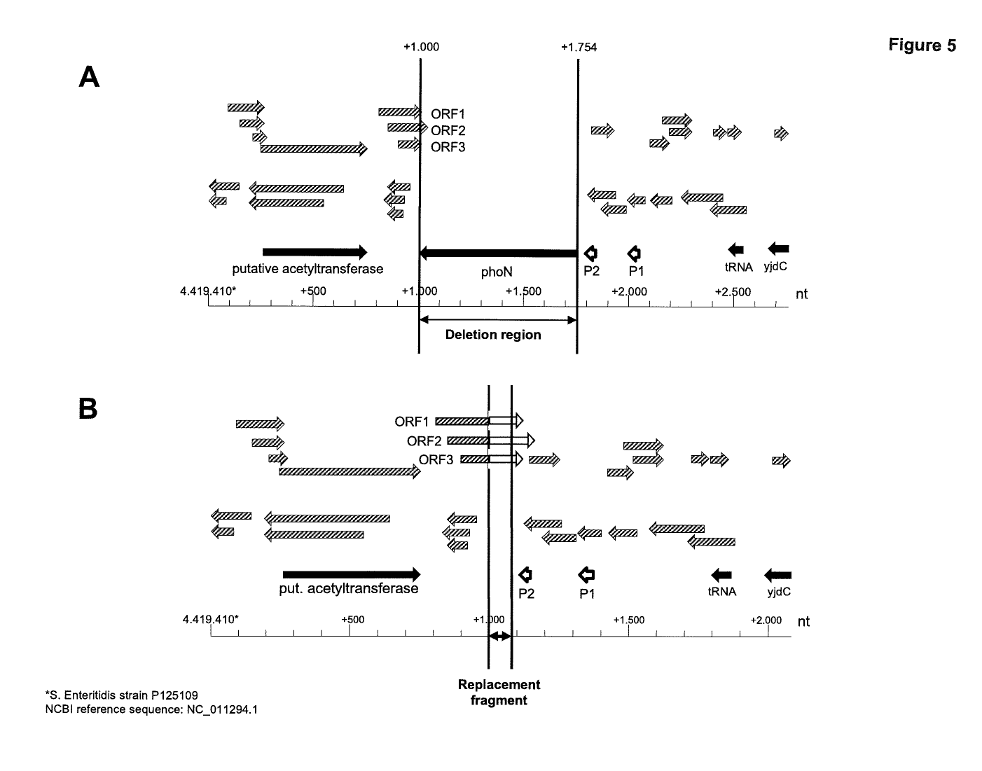

FIG. 5 exemplarily shows the strategy for the preparation of phoN deletion mutant of Salmonella enterica enterica Enteritidis strain P125109 by replacement recombination. In FIG. 6, RT-PCR analyses are presented which include the replacement fragment within the phoN deletion mutant of a Salmonella enterica enterica Enteritidis vaccine strain. The analyses revealed that the FRT-site within the replacement fragment unexpectedly acts as transcriptional terminator. Accordingly, external transcripts are ceased within the disclosed replacement fragment.

Finally, the inactivation process of the gene phoN may also include those genes or gene products that affect phoN activity, such as phoP and phoQ. The inactivation or deletion of phoP and/or phoQ in Salmonellae suppresses phoN activity. Accordingly, Salmonella with inactive or deleted phoP and/or phoQ may work like phoN-DIVA vaccine strain.

The outlined strategies can be applied for any live-attenuated Salmonella vaccine strain. The primary structure of the genomic region which encompasses the genetic engineering processes can be verified by DNA sequencing in order to define the correct structure of the deletion and flanking regions.

Discrimination of Live-Attenuated Salmonella phoN-DIVA Vaccine Strain from Wild Type Salmonella by Bacteriological Means.

The detection of Salmonella in livestock is primarily done by bacteriological methods. Most frequently the method according to ISO6579 is used for Salmonella detection.

The bacteriological phoN-DIVA test is preferentially performed on an agar plate, e.g. Luria Broth agar (LB agar), which contains a phosphatase specific substrate, e.g. 5-Brom-4-chlor-3-indoxylphosphat (BCIP) at concentrations of 40-80 μg/mL. BCIP is an uncolored compound which becomes converted into a blue-green colored product by phosphatases. Since Salmonella expresses several enzymes with phosphatase activity, it was surprising that none of the live-attenuated phoN-DIVA Salmonella vaccine strains was able to produce colored colonies on BCIP LB agar plates but wild type Salmonella do so (FIG. 7). Inactivation of phoN is sufficient to prevent the chromogenic phosphatase reaction described herein. The suspicious Salmonella isolate is dispersed on the BCIP LB agar plate and after incubation at 37° C. for 12-24 hours the appearance of colored or uncolored colonies is determined. The wild type Salmonella which expresses PhoN generates blue-green colored colonies. In the contrast, the live-attenuated Salmonella phoN mutant vaccine strain remains uncolored as indicated in FIG. 7.

The bacteriological phoN-DIVA-testing supplements the standard testing. It is applied whenever the standard method specifically looks for Salmonella, e.g. after vaccination of livestock with a live-attenuated Salmonella DIVA-vaccine strain.

Discrimination of Live-Attenuated Salmonella enterica enterica phoN-DIVA vaccine Strain from Wild Type Salmonella by Antibody Testing Using Samples of Vaccinated Animals.

Antibody testing is another routine method for the detection of Salmonella infected livestock. The antibody testing is primarily applied to screen livestock for previous Salmonella infections and to verify successful vaccination. However, the antibody tests on the market are not able to discriminate between Salmonella infected and Salmonella vaccinated animals.

In the following experiments the discriminating character of the protein PhoN for DIVA antibody testing is assessed. The approach was analysed at first in S. Typhimurium mouse model. One group of mice was orally immunized with live-attenuated Salmonella Typhimurium phoN-DIVA vaccine strain (SL1344 ΔaroA, ΔphoN). In a parallel group, susceptible mice (Nramp⁺) were orally infected with wild type Salmonella Typhimurium (SL1344). In both animal groups a strong humoral (IgG) immune response against S. Typhimurium lipopolysaccharide (LPS) was detected (FIG. 8). PhoN specific immune response was evaluated by immunoblotting using purified recombinant protein. For the testing two variants of recombinant PhoN, namely PhoN_{FP101} and PhoN_{FP201}, has been applied. As shown in FIG. 8B, mice infected with wild type S. Typhimurium do generate IgG antibodies against PhoN. In the contrast, mice orally vaccinated with live-attenuated Salmonella Typhimurium ΔphoN-DIVA vaccine strain do not generate antibodies against PhoN. These data clearly reveal that Salmonella infected mice produce antibodies which are highly specific for PhoN.

Secondly, the practicability of the approach for livestock is assessed. Young chicks were orally immunized with live-attenuated Salmonella Enteritidis ΔphoN-DIVA vaccine strain. Another group of chicks become orally infected with wild type S. Enteritidis e.g. S. Enteritidis 147N. In both animal groups high IgG titer, specific for S. Enteritidis lysate was evident (FIG. 9). However, only the infected chicks generate IgG specific for recombinant PhoN. Chicks vaccinated with live-attenuated Salmonella Enteritidis phoN-DIVA vaccine strain do not produce PhoN-specific antibodies (FIG. 9). Finally, the detection of PhoN-specific antibodies was also approved for pigs infected with S. Typhimurium (FIG. 9).

The data clearly demonstrate, that PhoN-DIVA antibody test has broad applicability in different animals and for different Salmonella serovar.

Preparation of Recombinant PhoN Protein for PhoN-DIVA Antibody Test.

PhoN-DIVA antibody test is preferentially prepared on the basis of recombinant PhoN. Previous immunoblot analyses with purified recombinant PhoN clearly reveal that animals vaccinated with live-attenuated Salmonella DIVA-vaccine strain do not generate antibodies which bind to the whole protein. It was surprising that no cross-reactive antibodies do exist within sera of Salmonella-vaccinated animals, despite the complexity of the protein PhoN and the broad-spectrum reactivity of the humoral immune response.

The recombinant PhoN for PhoN-DIVA antibody test may be prepared with high purity. Any impurities may be detected by the broad-spectrum antibodies of vaccinated livestock and thus cause false results in the PhoN-DIVA antibody test.

The preparation of pure PhoN may be facilitated by extending the primary structure of PhoN with an extra polypeptide fragment that allows highly stringent purification conditions. Preferentially, a series of histidine (His-tag) is introduced either at the amino terminus or at the carboxy terminus of PhoN. Furthermore, another protein domain may be introduced between the His-tag and the PhoN structure which is specifically recognized by an endopeptidase, e.g. thrombin, which allows the removal of the His-tag. The analyses reveal that such a fusion protein of PhoN which comprises the His-tag and thrombin-site does not interfere with sera of animals vaccinated with live-attenuated Salmonella DIVA-vaccine strain as demonstrated in the previous Figures.

The fusion proteins of PhoN can be prepared in Escherichia coli by conventional expression vector, e.g. pET15b and by applying standard purification protocol as indicated by the supplier, e.g. Novagen. FIGS. 10-12 disclose the maps of the used expression plasmids, the DNA and the amino acid sequences of the exemplarily prepared PhoN fusion proteins, PhoN_{FP101} and PhoN_{FP201}.

PhoN-DIVA Antibody Test.

PhoN-DIVA antibody test is applied whenever livestock becomes vaccinated with live-attenuated Salmonella phoN-DIVA vaccine strain. In this example, the serological PhoN-DIVA test system comprises the recombinant protein PhoN, e.g. PhoN_{FP101} or PhoN_{FP}₂₀₂, and another Salmonella specific antigen. Preferentially, the Salmonella specific antigen is provided by a commercial test kit which detects Salmonella specific antibodies in chicken and pigs, e.g. provided by Idexx Laboratories Inc. (Herdchek, Flockchek) or by Labor Diagnostik GmbH Leipzig (Flocktype Salmonella, Salmotype Pig Screen) or by Biochek and others. This test set-up proves whether Salmonella specific immune response appears after vaccination. Additionally, it proves that no wild-type Salmonella was involved which is indicated by negative reaction in the PhoN-DIVA antibody test.

Generally, the commercial test kits for the detection of Salmonella specific antibodies are prepared as an indirect ELISA in microtiter plates. Depending on the provider, the Salmonella-specific antigen or antigen mix is already bound to the vials of the microtiter plate or must be coupled later on using an aliquot of a stock solution.

The purified PhoN, e.g. PhoN_{FP201}, is preferentially provided as a frozen stock solution or as a lyophilisate with appropriate preservative agents. In this Example, the ELISA for the detection of PhoN-specific antibodies is performed according to standard protocol as described elsewhere, e.g. by E. Harlow and D. Lane in Antibodies. A Laboratory Manual.

Production of Live-Attenuated phoN-DIVA Salmonella Vaccine Strain and Vaccination Protocol.

As already indicated the phoN-DIVA approach can be applied to any known and future live-attenuated Salmonella vaccine strain. The inactivation of phoN does not significantly affect the immunogenic potential of the appropriate Salmonella vaccine strain. Accordingly, after transformation of the Salmonella vaccine strain into a phoN-DIVA vaccine strain no changes are needed, neither for the production process nor for the vaccination protocol.

1. A Salmonella strain having an inactivated phoN gene.

2. The strain of claim 1, which is an attenuated Salmonella strain.

3. The strain of claim 1 or 2 for use in medicine, e.g. in veterinary medicine.

4. The strain of claim 3 for use as a vaccine.

5. The strain of claim 4 for use as a live vaccine.

6. The strain of claim 4 or 5 for use as a vaccine to protect against a Salmonella infection.

7. The strain of claims 1-6, which is a live-attenuated Salmonella vaccine strain, particularly
(i) a vaccine strain prepared by chemical mutagenesis and/or genetic engineering,
(ii) a recombinant vaccine strain which optionally recombinantly expresses at least one autologous Salmonella antigen and/or at least one heterologous antigen, e.g. an antigen of a heterologous pathogen, or
(iii) a recombinant carrier strain for the delivery of DNA.

8. The strain of any one of claims 1-7 which is a Salmonella Enteritidis ΔphoN His⁻ Ade⁻ strain.

9. The strain of any one of claims 1-8 for use in mammals, e.g. pigs, or birds, e.g. poultry such as chickens.

10. Use of a polypeptide comprising a Salmonella phoN polypeptide or/and any immunogenic portion thereof as a serological marker antigen, wherein the immunogenic portion has a length of preferably at least 10, more preferably at least 20 and even more preferably at least 30 amino acids.

11. The use of claim 10 for the differentiation of infected from vaccinated animal (DIVA) after vaccination with the strain of any one claims 1-8.

12. The use of claim 10 or 11, wherein the phoN antigen is prepared by recombinant expression of a phoN nucleic acid, particularly in a recombinant host cell such as E.coli.

13. The use of any one of claims 10-12, wherein the phoN antigen comprises the full-length phoN polypeptide.

14. A serological test system for detecting antibodies directed against a Salmonella phoN antigen, comprising at least one recombinant Salmonella phoN antigen or any immunogenic portion thereof and optionally further test components.

15. Use of a Salmonella phoN nucleic acid for the manufacture of a recombinant Salmonella phoN antigen or any immunogenic portion thereof.
